(19) United States
(12) Patent Application Publication
(10) Pub. No.: US 2020/0197447 A1
(43) Pub. Date: Jun. 25, 2020

(54) BACTERIOTHERAPY BASED ON BACTERIAL COMPOSITIONS FOR THE TREATMENT OF NEURODEGENERATIVE DISEASES

(71) Applicant: PROBIOTICAL S.p.A., Novara (IT)

(72) Inventor: Giovanni MOGNA, Novara (IT)

(21) Appl. No.: 16/615,355

(22) PCT Filed: May 23, 2018
(86) PCT No.: PCT/IB2018/053632

§ 371 Date: Nov. 20, 2019

(30) Foreign Application Priority Data
May 25, 2017 (IT) 102017000057079

(51) Int. Cl. A61K 35/74; A61P 25/28

(52) U.S. Cl. CPC A61K 35/74; A61P 25/28

(57) ABSTRACT

The present invention relates to a bacteriotherapy based on bacterial compositions for the treatment and/or the prevention of neurodegenerative diseases, wherein said composition comprises an effective amount of bacterial microorganisms belonging to the species Lactobacillus fermentum, Lactobacillus delbrueckii subsp. delbrueckii, Lactobacillus plantarum and Lactobacillus salivarius, and mixtures thereof.

The present invention relates to a bacteriotherapy based on bacterial compositions for the treatment and/or the prevention of neurodegenerative diseases, wherein said composition comprises an effective amount of bacterial microorganisms belonging to the species Lactobacillus fermentum, Lactobacillus delbrueckii subsp. delbrueckii, Lactobacillus plantarum and Lactobacillus salivarius, and mixtures thereof.

Amyotrophic Lateral Sclerosis (ALS) is a particularly devastating neurodegenerative disease which affects motoneurons and their connections with muscles, for which no therapy exists and which leads to death due to respiratory failure within 2-5 years after the onset of symptoms. Neuronal loss in ALS is a much more rapid process than the one that occurs in other degenerative pathologies (Kanazawa I. “How do neurons die in neurodegenerative diseases?” Trends Mol Med. 2001; 7(8):339-44). Despite the improvement in knowledge about the pathogenetic mechanisms that lead to cell death, ALS continues to represent one of the great enigmas of neurosciences. The aetiology of ALS is unknown and the pathogenesis remains unclear. In 90% of the cases it is sporadic, whilst in 10% of the cases there is a hereditary factor and about 30 genes linked to the disease have been identified to date. It is believed to be a multifactorial disease in which different predisposing genes interact with environmental factors in the manifestation of the disease.

Recent data have demonstrated that nutritional state represents an independent prognostic factor for the survival of patients with ALS (Gallo V. et al “Prediagnostic body fat and risk of death from amyotrophic lateral sclerosis: the EPIC cohort” Neurology. 2013, 80(9):829-38). During the course of the disease or in some cases even prior to its onset a hypercatabolic state arises, whose origin is uncertain. The presence of hypercatabolism is paradoxical, since the disease, by leading to muscular atrophy, typically brings about a loss in lean body mass, and lean body mass is the main factor determining energy consumption or REE (resting energy expenditure). The literature has suggested, therefore, that these patients are not intrinsically hypercatabolic, but during the progression of the disease factors such as respiratory failure, with an increase in the work of accessory respiratory muscles, progressive motor disability with a compensatory increase in the work of residual muscles, or, additionally, a chronic inflammatory state due to infections, including silent ones, may contribute to the increase in calorie consumption. However, recent studies have called these data into question by demonstrating an absence of correlations between hypermetabolism and respiratory function in a sample of ALS patients, while the hypercatabolic state correlates with age, sex and percentage of lean body mass. It is thus hypothesised that other factors linked to the disease itself and not to the functional situation may come into play.

The role of neuroinflammation through microglial activation and reactive astrocytes in the genesis and evolution of ALS is demonstrated in numerous studies (Boillee S, et al. “ALS: a disease of motor neurons and their not neuronal neighbours” Neuron 2006; 52:39-59; Boillee S, et al. “Onset and progression in inherited ALS determined by motor neurons and microglia” Science 2006, 312:1389-1392).

Some strains of Clostridium can produce neurotoxins that selectively affect the motor system. The best known strains are the ones that cause botulism or tetanus, but other strains such as Clostridium baratii and Clostridium butyricum produce neurotoxins, whose effect on the nervous system is not entirely clear.

A study conducted by S. Wu et al., “Leaky intestine and impaired microbiome in an amyotrophic lateral sclerosis mouse model” Physiol Rep, 3(4), 2015, e12356, seems to suggest a potential role of the intestinal epithelium and the microbiome in the progression of ALS.

In a study conducted by Y. Zhang et al., “Target Intestinal Microbiota to Alleviate Disease Progression in Amyotrophic Lateral Sclerosis” Clinical Therapeutics, Vol. 39, No. 2, 2017, mice affected by ALS were treated with sodium butyrate. The study suggests that treatment with butyrates seems to restore the homeostasis of the intestinal microbiota.

One object of the present invention relates to a composition for use in the treatment of neurodegenerative diseases, such as ALS, which is easy to administer, easily tolerable, i.e. substantially free of significant side effects, and of natural origin.

Another object of the present invention relates to a method or treatment regime that provides for the administration of a first and a second composition for use in the treatment of neurodegenerative diseases, such as ALS, which are easy to administer and easily tolerable, i.e. substantially free of significant side effects.

The subject matter of the present invention is a composition and a method (or treatment regime) having the features as defined in the appended independent claims for use in the treatment of at least one degenerative disease.

Preferred embodiments of the present invention will become apparent from the detailed description that follows and are claimed in the appended claims.

In the context of the present invention the term “composition(s)” relates to a pharmaceutical composition, or a composition for a medical device, or a composition for a dietary supplement, or a food composition.

Following extensive experimentation, the inventors have developed a composition useful for the curative and/or preventive treatment of neurodegenerative diseases, such as ALS, containing bacterial strains.

Within the scope of the present invention, “treatment” of neurodegenerative diseases means therapy aimed at improving the health conditions of a subject, maintaining the existing conditions and/or preventing the rapid worsening (slowing down the worsening) of said health conditions.

Within the scope of the present invention, “prevention” of neurodegenerative diseases means therapy aimed at avoiding the onset of such a disease in an subject, also, but not only, as a complication or effect of a pre-existing pathological condition or disorder.

“Neurodegenerative disease” means a pathology of the central or peripheral nervous system, characterised by a chronic, selective process of cell death or severe degeneration affecting neurons. Non-limiting examples of neurodegenerative diseases are Alzheimer's disease, Parkinson's disease, Amyotrophic Lateral Sclerosis (ALS) or Lou Gehrig's disease, senile dementia, Huntington's disease, progressive supranuclear palsy, frontotemporal dementia and Lewy body dementia.

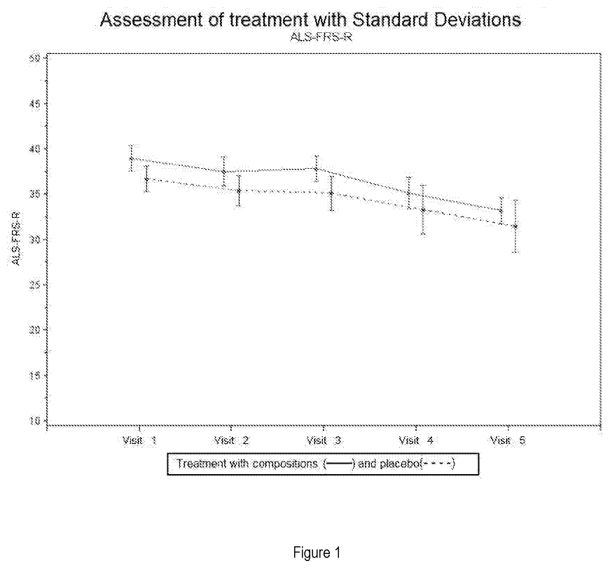

FIG. 1 shows the effect of treatment with the composition of the invention (vs placebo) on the course of the disease disability measured with the ALS-FRS-R scale. The ALS-FRS-R (IR 25%) parameter is a parameter used by clinicians to define patients' state of health.

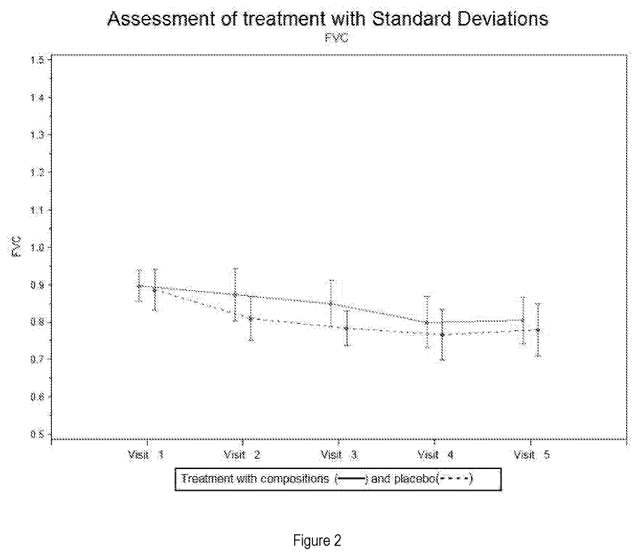

FIG. 2 shows the effect of treatment with the composition of the invention (vs placebo) on the course of the disease in terms of forced vital capacity (FVC).

In one aspect, the present invention provides a composition comprising an effective amount of a mixture which comprises or, alternatively, consists of at least one bacterial strain, for use in the preventive and/or curative treatment of the symptoms and/or disorders connected to at least one neurodegenerative disease in a subject, wherein said use comprises the administration of the composition to said subject; said subject is preferably affected by ALS.

In the composition for use according to the present invention, said at least one bacterial strain preferably belongs to the genus Lactobacillus or the genus Streptococcus, more preferably it is selected from among the species Streptococcus thermophilus, Lactobacillus fermentum, Lactobacillus delbrueckii, Lactobacillus delbrueckii subspecies delbruceckii, Lactobacillus plantarum and Lactobacillus salivarius or mixtures thereof.

In an even more preferred embodiment, said at least one bacterial strain is selected from the group comprising or, alternatively, consisting of (I) Streptococcus thermophilus ST10 deposited by Probiotical SpA with the Institute DSMZ in Germany on 19 Sep. 2011 and having the deposit number DSM 25246, (11) Lactobacillus fermentum LF10 deposited by Anidral Srl (now Probiotical SpA) with the Institute DSMZ in Germany on 20 Mar. 2007 and having the deposit number no. DSM 19187, (111) Lactobacillus delbrueckii subsp. delbrueckii LDD01 deposited by Probiotical SpA with the Institute DSMZ in Germany on 10 Dec. 2008 and having the deposit number no. DSM 22106, (IV) Lactobacillus plantarum LP01 deposited by Mofin Srl with the Institute BCCM LMG in Belgium on 16 Oct. 2001 and having the deposit number no. LMG P-21021, (V) Lactobacillus salivarius LS03 deposited by Probiotical SpA with the Institute DSMZ in Germany on 23 Jul. 2009 and having the deposit number no. DSM 22776 and, optionally, (VI) L. fermentum LF18 deposited by Probiotical SpA with the Institute DSMZ in Germany on 30 Jul. 2014 and having the deposit number no. DSM 29197 and (VII) L. fermentum LF19 deposited by Probiotical SpA with the Institute DSMZ in Germany on 30 Jul. 2014 and having the deposit number no. DSM 29198 and mixtures thereof.

All the bacterial strains were deposited in accordance with the provisions of the Budapest Treaty.

The set of the aforesaid bacterial strains (I) to (IV) is capable of exerting a significant anti-inflammatory activity, of opposing the secretion of inflammatory cytokines and inducing the secretion of regulatory and anti-inflammatory cytokines by cells of gut-associated lymphoid tissue. Furthermore, such microorganisms perform a barrier action against both yeasts of the genus Candida, and bacterial groups potentially implicated in the genesis, worsening and/or persistence of the typical symptoms of ALS, with particular reference to Clostridia and several coliforms. The set of the aforesaid bacterial strains exerts an anti-inflammatory activity combined with an inhibiting action against yeasts of the genus Candida. For this reason, said set of bacterial strains is capable of directly aiding in restoring the physiological intestinal barrier assured by the muco-adhering gelling complex, thus hindering the absorption of toxins, pro-inflammatory molecules and allergens which maintain a chronic hyper-reactivity of the immune cells in gut-associated lymphoid tissue (GALT) and, more in general, at a systemic level.

In detail, the bacterial strain L. fermentum LF10 deposited by Anidral Srl (now Probiotical SpA) with the Institute DSMZ in Germany on 20 Mar. 2007 and having the deposit number no. DSM 19187 exerts a particular action (and, optionally, in synergy with L. fermentum LF18 deposited by Probiotical SpA with the Institute DSMZ in Germany on 30 Jul. 2014 and having the deposit number no. DSM 29197 and L. fermentum LF19 deposited by Probiotical SpA with the Institute DSMZ in Germany on 30 Jul. 2014 and having the deposit number no. DSM 29198, if present) of opposing the yeasts belonging above all to the genus Candida, with particular reference to the five species C. albicans, C. glabrata, C. parapsilosis, C. tropicalis and C. krusei. Furthermore, the bacterial strain L. fermentum LF10 induces the secretion of regulatory and anti-inflammatory cytokines (for example, IL-10) by GALT.

The bacterial strain L. delbrueckii subsp. delbrueckii LDD01 deposited by Probiotical SpA with the Institute DSMZ in Germany on 10 Dec. 2008 and having the deposit number no. DSM 22106 exerts a strong anti-inflammatory activity of inducing the secretion of regulatory and anti-inflammatory cytokines by GALT and an antagonistic action against various genera of intestinal bacteria, above all coliforms (E. coli and Klebsiella pneumoniae) and Clostridium difficile.

The bacterial strain L. plantarum LP01 deposited by Mofin Srl with the Institute BCCM LMG in Belgium on 16 Oct. 2001 and having the deposit number no. LMG P-21021 exerts a significant anti-inflammatory activity as it produces, among other things, IL-10 and aids in the inhibiting activity against coliforms, with particular reference to E. coli.

The bacterial strain L. delbrueckii subsp. delbrueckii LDD01 deposited by Probiotical SpA with the Institute DSMZ in Germany on 10 Dec. 2008 and having the deposit number no. DSM 22106 and the bacterial strain L. plantarum LP01 deposited by Mofin Srl with the Institute BCCM LMG in Belgium on 16 Oct. 2001 and having the deposit number no. LMG P-21021 together exert a strong action against intestinal dysbiosis.

Finally, the bacterial strain L. salivarius LS03 deposited by Probiotical SpA with the Institute DSMZ in Germany on 23 Jul. 2009 and having the deposit number no. DSM 22776, exerts an important immunoregulatory activity.

In a preferred embodiment, the method of treatment by means of the composition according to the present invention comprises the daily administration of an amount comprised from 1×10⁹ to 1×10¹², preferably from 1×10¹⁰ to 1×10¹¹, viable cells per strain per dose, divided into one, two or three doses a day.

In one embodiment the mixture, which is contained in said composition of the present invention, comprises or, alternatively, consists of the five bacterial strains (I) to (V) in a weight ratio of 1:1:1:1:1.

In one embodiment, the administration of the composition to the subject takes place orally, for example in the form of a pill, tablet, which may also be coated, granules or powder to be reconstituted or dissolved in the mouth, for example in sachets or stick packs, a solution, suspension, syrup, food containing the bacterial strains, or in any other form known to the person skilled in the art.

It remains understood that, if the treatment according to the invention comprises the administration of more than one bacterial strain, said administration according to the invention can take place simultaneously, for example in a single formulation (all the bacterial strains together), or in a rapid sequence, for example with two or more formulations taken by the subject in any order, in a sequence closely spaced in time (e.g. within 1 to 10 minutes) in two distinct compositions.

The composition for use according to the present invention can comprise, in addition to one or more bacterial strains, at least one inert ingredient, such as at least one excipient among the ones commonly used and known to the person skilled in the art.

“Inert ingredient” means any substance, or combination of substances, auxiliary to the production of a pharmaceutical, dietary or nutraceutical form, which is to be found in the finished product and is not the active ingredient, although it can modify the stability, release or other characteristics thereof. Non-limiting examples of said inert ingredient, as is known to the person skilled in the art of formulations in the pharmaceutical, nutraceutical or food industry, are excipients such as diluents, absorbents, lubricants, colourants, surfactants, antioxidants, sweeteners, binders, disaggregating agents and the like.

In one embodiment, the composition for use according to the present invention comprises, in addition to one or more bacterial strains, at least one further compound of natural or synthetic origin. Non-limiting examples of said compounds are vitamins, antioxidants, or vegetable substances and preparations (botanicals).

In a preferred embodiment, the composition for use according to the present invention contains, in addition to said at least one bacterial strain, at least one vegetable gum and/or an animal and/or vegetable gelatine.

A gum is a dehydrated or lyophilised or dried material in the form of a powder or flakes which on coming into contact with water produce a gum gel in water (aqueous gel) or a gum gelatine. Alternatively, an already prepared gel or gelatine may be validly used.

In one embodiment the vegetable gum and/or animal and/or vegetable gelatine is selected from the group comprising or, alternatively, consisting of Aloe, such as Aloe vera (or Aloe barbadensis Miller, a plant of the family Aloeacee) or Aloe arborescens, alginates, xyloglucans (or xylogels), carrageenans, pectins and agar-agar.

In one embodiment, the composition according to the invention comprises a gum based on galactomannans, such as tara gum, guar gum or other gums suitable for dietary, nutraceutical and/or pharmaceutical use; the composition according to the present invention preferably comprises tara gum (e.g. obtained from ground seeds of Caesalpinia spinosa), indicated as E471, in association with the specified bacterial strains (I) to (VI).

In one embodiment of the present invention, the treatment of a subject affected by a neurodegenerative disease, such as ALS, comprises, in addition to the administration of the composition as described above (said first composition of the present invention), the administration of a second composition such as, for example, a dietary supplement (said second composition of the present invention) comprising at least one substance selected from the group comprising or, alternatively, consisting of (i) Artemisia vulgaris, (ii) Carum carvi, (iii) Lavender angustifolia, otherwise known as Lavandula officinalis, (iv) Mentha piperita, and mixtures thereof.

Said supplement (second composition) preferably contains at least four active ingredients of vegetable origin specifically selected so as to reduce, in a wholly natural manner, the intestinal concentration of microbial groups and genera potentially implicated in the onset, persistence and/or worsening of the typical symptoms of ALS. Said second composition is preferably administered to a subject affected by a neurodegenerative disease for an initial continuous period ranging from 1 to 4 weeks, preferably from 2 to 3 weeks. Subsequently, or simultaneously, the composition of the present invention (said first composition of the present invention) is also administered to the same subject for a continuous period of time ranging from 4 to 16 weeks, preferably 12 weeks or three months. In this manner, it is also possible to enhance the effectiveness of the composition of the present invention administered in the three successive months of treatment with said second composition (supplement).

The set of 4 vegetable preparations, preferably in a weight ratio of 1:1:1:1, exerts a selective antibacterial, antifungal and antiparasitic activity. Among the selected oils, lavender and cumin (Carum carvi) represent the most important phytocomplexes with bactericidal activity. In particular, Carum carvi (cumin) is highly active, probably by virtue of its terpenic component, against Enterobacteriaceae, with particular reference to various species of Shigella, Salmonella and Yersinia (Moon, 2006). The two oils also have particular anti-fermentative and carminative properties. Lavender, thanks to the presence of a high percentage of polyphenols, also has a good action against the family Clostridiaceae, various species of Campylobacter and several saprophytic microorganisms with a marked putrefactive action, such as Enterobacter spp. and Enterococcus spp. As regards the fungal realm, among the main strains involved in intestinal immune activation, mention can be made of Mucor spp., aspergilli, penicilliums and above all Candida. The oils present in the dietary supplement of the present invention (said second composition of the present invention) include lavender oil, considered highly effective because of its direct antibiotic effect on fungi. The other important essential oil is mint oil, with a considerable action against the majority of aspergilli and penicilliums. Mint, furthermore, has parasympathicotonic properties: the resulting stimulation of the autonomous nervous system greatly improves digestive capacities (D'Auria, 2005). The essential oil that provides the most important action in the parasitological field (in terms of both protozoa and helminths) is Artemisia vulgaris.

The association of the 4 active ingredients (i) to (iv) specified above is capable of exerting a joint antibacterial, antifungal and antiparasitic activity.

Said supplement (said second composition of the present invention) is in liquid form, for example a solution having a volume comprised from 5 ml to 50 ml, preferably from 10 ml to 25 ml. Said second composition comprises a stock solution of all 4 of the above-mentioned active ingredients in the following concentrations: (i) Artemisia vulgaris, from 200 mg/50 ml to 600 mg/50 ml, for example 400 mg/ml; (ii) Carum carvi, from 150 mg/50 ml to 550 mg/50 ml, for example 350 mg/50 ml; (iii) Lavender angustifolia, otherwise known as Lavandula officinalis, from 450 mg/50 ml to 850 mg/50 ml, for example 650 mg/50 ml and (iv) Mentha piperita, from 400 mg/50 ml to 800 mg/50 ml, for example 600 mg/50 ml. All 4 active ingredients (i) to (iv) are preferably present in a 1:1:1:1 ratio.

The subject matter of the present invention relates to a composition comprising a mixture which comprises or, alternatively, consists of at least one bacterial strain in an effective amount for the treatment and/or prevention of a neurodegenerative disease.

The subject matter of the present invention further relates to a method (or treatment regime) which comprises the administration of said first and second compositions for the treatment and/or prevention of a neurodegenerative disease.

In the composition of the present invention, said at least one bacterial strain is preferably selected from the group comprising or, alternatively, consisting of (I) Streptococcus thermophilus ST10 deposited by Probiotical SpA with the Institute DSMZ in Germany on 19 Sep. 2011 and having the deposit number no. DSM 25246, (II) Lactobacillus fermentum LF10 deposited by Anidral Srl (now Probiotical SpA) with the Institute DSMZ in Germany on 20 Mar. 2007 and having the deposit number no. DSM 19187, (111) Lactobacillus delbrueckii subsp. delbrueckii LDD01 deposited by Probiotical SpA with the Institute DSMZ in Germany on 10 Dec. 2008 and having the deposit number no. DSM 22106, (IV) Lactobacillus plantarum LP01 deposited by Mofin Srl with the Institute BCCM LMG in Belgium on 16 Oct. 2001 and having the deposit number no. LMG P-21021, (V) Lactobacillus salivarius LS03 deposited by Probiotical SpA with the Institute DSMZ in Germany on 23 Jul. 2009 and having the deposit number no. DSM 22776 and, optionally, (VI) L. fermentum LF18 deposited by Probiotical SpA with the Institute DSMZ in Germany on 30 Jul. 2014 and having the deposit number no. DSM 29197 and (VII) L. fermentum LF19 deposited by Probiotical SpA with the Institute DSMZ in Germany on 30 Jul. 2014 and having the deposit number no. DSM 29198 and mixtures thereof.

In one preferred embodiment, each of the bacterial strains can be present in the composition of the present invention in an amount comprised from 1×10⁹ to 1×10¹², preferably from 1×10¹⁰ to 1×10¹¹ viable cells per strain.

More preferably, said composition further comprises at least one vegetable gum, such as tara gum, and/or at least one animal gelatine.

The composition of the present invention can be solid, for example in the form of a powder or granules to be reconstituted, or in a liquid or semisolid form to be dissolved in the mouth, such as, for example, a suspension or gel, and can be in any form of administration known the person skilled in the art, such as, by way of non-limiting example, in the form of a capsule, tablet, or powder that is at least partially dissolvable in the mouth or water soluble, granules, pellets or microparticles and optionally contained in a sachet, in a stick pack or in a capsule or mini-tablet, liquid or semisolid preparation, gel, suspension, solution, two-phase liquid system and equivalent forms.

The following experimental part provides examples of practical embodiments of the invention, without limiting the scope thereof.

### Experimental Part

For the purpose of assessing the effectiveness of specific selected bacterial strains in neurodegenerative diseases, such as in ALS, preliminary investigations were conducted on peripheral blood polymorphonuclear cells.

The in vitro study conducted by the inventors aimed to analyse the effects of selected strains of microorganisms on the main cellular subpopulations involved in the innate and acquired immune responses.

The following in vitro trials were conducted.

Tests were performed on the strains grown to confluence in 24-well plates of CACO-2, in vitro model of intestinal epithelial tissue, in order to assess the effect of repair or protection against pro-inflammatory stimuli (TNF-alpha and IL-1 beta).

The cell populations of interest (PBMC—peripheral blood mononuclear cells) were isolated using a standard method (Dextran and Ficoll centrifugation) from samples of venous blood from healthy donors and/or Parkinson or ALS patients.

For the purpose of determining which cellular subpopulations were induced to proliferate following stimulation with the selected strains, a multi-parameter cytofluorimetric analysis was performed. The main cellular subpopulations involved in the innate and acquired immune response were investigated.

The concentration of cytokines was determined by E.L.I.S.A. (Enzyme-Linked Immunoabsorbent Assay). Cytokines such as TNF-α, IFN-γ, IL-1beta, IL-18, IL17A and type Th2 cytokines, mainly IL-10, were investigated.

An evaluation of oxidative stress was conducted on the patients' plasma with an in vitro model of hyperhomocysteinaemia evaluated by spectrophotometry.

### Clinical Study to Determine the Effectiveness and Tolerability of the Treatment According to the Present Invention

### Objectives

The main objective was to quantify the concentrations of several species of intestinal bacteria in patients with amyotrophic lateral sclerosis (abbreviated as ALS) compared to healthy subjects in order to subsequently evaluate the influence/modification that could be generated as a result of a treatment with a composition of the present invention comprising an association of a natural gelling complex (EPS—exopolysaccharides produced in situ by bacteria) with specific lactobacilli in the concentrations of said species of intestinal bacteria and to be able to have a correlation with the disability measured with the ALS-FRS-R scale (e.g. according to Cedarbaum, J. M. et al. Journal of the Neurological Sciences 1999, 169, 13-21) and spirometry, which is used to measure, among other things, FVC (“forced vital capacity”), a parameter that represents a diagnostic index of fundamental importance in clinical medicine (e.g. according to Quanjer, Philip H. et al. GLI-2012, 1-16, reference values for spirometry). The parameter ALSFRS (Amyotrophic Lateral Sclerosis Functional Rating Scale) is a validated scale based on specific questions that measures physical function in carrying out activities of daily living (ADL) in patients with ALS.

The study also aimed to: a) assess whether the levels of intestinal bacteria have a significant association with nutritional state in patients with ALS and whether treatment with a composition of the present invention modifies it; b) assess whether the treatment with the composition according to the invention influences the quality of life of patients.

### Patients and Methods

Study Design.

It was a single-centre, phase II, randomised, double-blind, placebo-controlled clinical trial.

### Subjects.

The project provided for the enrolment in 1 year of 50 subjects (30 men and 20 women) with ALS admitted to a neurological clinical centre specialised in ALS.

Inclusion criteria: diagnosis of ALS, definite or probable according to El Escorial criteria; age ranging between 18 and 75 years; duration of the disease (since the time of diagnosis)<3 years; FVC (forced vital capacity) >50%.

Exclusion criteria: patients affected by concomitant pathologies (gastrointestinal tract diseases, motor neuron diseases other than ALS, clinical involvement of other neurological systems, malign neoplasia, inflammatory and autoimmune diseases, serious cardiocirculatory and respiratory system diseases); family history of ALS; patients with PEG or nasogastric tubes; tracheotomy or non-invasive ventilation for a period >18 hours; intake of drugs or lactobacilli that may have modified the intestinal bacterial flora in the 8 weeks preceding recruitment; women who are pregnant or may become pregnant during the treatment, participation in experimental therapies in the 3 months prior to enrolment, patients unable to understand the informed consent form.

### Assessment

All patients underwent a neurological assessment including completion of the forms based on the ALS—FRS-R scale, spirometry and measurement of forced vital capacity (FVC %) and expiratory volume (FEV1) as well as completion of the ALSAQ-40 questionnaire with scales for measuring quality of life. All patients further underwent a dietary assessment comprising: assessment based on medical history and objective assessment of the impairment in the ability to feed oneself autonomously, chewing and swallowing; assessment of weight loss compared to premorbid weight; dietary history (assessment of average calorie and protein intake over 24 hours); measurement of height and weight; calculation of the body mass index (BMI)=weight [kg]/height [m]2. All personal and clinical data and medical history were recorded in a datasheet.

### Randomisation

Patients who met the inclusion criteria were randomised by an independent statistician responsible for assigning a code number generated by a computerised program. The ratio was 1:1. A first and a second group of subjects were thus formed (treated group and control or placebo group). After a run-in period of 1 month a first group of subjects (treated group) received, for 20 consecutive days (first treatment stage), a dietary supplement (said second composition of the present invention) containing an association of 4 natural active ingredients of vegetable origin with an antibacterial, antifungal and anthelmintic activity. Whereas the second group of subjects (control or placebo group) received an amount of 10 ml in volume of a placebo in a form that was indistinguishable from said dietary supplement. At the end of the first treatment stage, the first group of subjects also received a composition of the present invention, in solid form, based on an association of a specific gelling complex (EPS—exopolysaccharides) produced in situ by the bacterial strain (I) Streptococcus thermophilus ST10 deposited by Probiotical SpA with the Institute DSMZ in Germany on 19 Sep. 2011 and having the deposit number no. DSM 25246, once administered, and the 4 selected bacterial strains indicated as (II) to (V) for 3 months (second treatment stage). Whereas the second group of subjects received an equal amount of an inert substance in a form indistinguishable from the composition of the present invention. At the end of the third month of treatment, the randomisation codes were disclosed and the subjects in the placebo group could begin treatment with the dietary supplement containing the 4 natural active ingredients of vegetable origin (active ingredients (i) to (iv)) followed by oral administration of the composition of the present invention (bacterial strains (I) to (V)), whereas the subjects in the first group (treated group) could continue taking the bacterial strains on a continuous basis “sine die” for maintenance purposes. In the study protocol, administration for the following 3 months was provided for, but patients continued taking the supplement for an indefinite period.

### Procedures

1. After being informed of the aims of the study and signing an informed consent form, the patient was asked to provide a faecal sample. Sampling was repeated at the end of the 3rd month of treatment with the composition of the present invention or with the placebo. A further sample was collected 6 months after recruitment in both groups.

2. The assay of specific microbial groups and species was carried out according to a specific standardised protocol based on the extraction of total DNA and subsequent quantification by fluorescent in situ hybridization (FISH). The samples were analysed in triplicate using an epifluorescence microscope. The data were expressed as number of cells per g of faeces.

### Assay of Bacteria

The faecal samples were immediately collected on a blind basis in sterile test tubes, frozen at minus 20° C. and subsequently used to quantify the bacteria belonging to the genera/families Clostridium, Enterobacteriaceae, total coliforms and lactobacilli (Franks A. H. et al. “Variations of bacterial populations in human feces measured by fluorescent in situ hybridization with group-specific 16S rRNA-targeted oligonucleotide probes” Appl Environ Microbiol 1998 64(9):3336-45; Harmsen H. J. M. “A 16S rRNA-targeted probe for detection of lactobacilli and enterococci in faecal samples by fluorescent in situ hybridization” Microb Ecol Health Dis. 1999, 11:3-12).

In addition to the above-mentioned bacterial groups, total yeasts were also quantified. In particular, within the genus Clostridium, a quantification was made of the species C. difficile and cluster I/II, the most representative species of which are C. baratii, C. hystoliticum, C. butyricum, C. prefringens, C. botulinum and C. tetani (Collins M. D. et al. “The phylogeny of the genus Clostridium: proposal of five new genera and eleven new species combinations” Int J Syst Bacteriol. 1994; 44(4):812-26; 4. Bloedt K. et al. “Evaluation of new selective culture media and a rapid fluorescence in situ hybridization assay for identification of Clostridium difficile from stool samples” J Med Microbiol 2009; 58(Pt 7):874-7; Langendijk P. S. et al. “Quantitative fluorescence in situ hybridization of Bifidobacterium spp. with genus-specific 16S rRNA-targeted probes and its application in fecal samples” Appl Environ Microbiol 1995; 61(8):3069-75).

The protocol followed comprised, in short, the following steps: the faecal samples were thawed and diluted 1:10 (w/v) in sterile PBS 0.1 M at pH 7.0. The test tubes were centrifuged at 2000×g for 2 minutes and aliquots of the faecal slurry were fixed in 4% paraformaldehyde overnight at 4° C. Following fixation, the cells were washed with PBS, centrifuged for 5 minutes at 13,000×g, resuspended in a PBS/ethanol solution (1:1) and preserved at −20° C. for at least 4 hours prior to hybridisation.

The bacterial populations were quantified by FISH analysis using oligonucleotide probes marked at 5′ with a fluorochrome and specific for the gene encoding rRNA 16S.

The hybridised cells were washed and fixed under vacuum on filters for the subsequent enumeration using an epifluorescence microscope. Fifteen random microscopic fields were counted for each test and used to calculate the number of cells on every filter.

The final data were expressed as number of cells per g of faecal material.

### Storage/Use of the Biological Material Collected for the Study

The biological samples were stored exclusively for the time necessary to carry out the planned analyses.

### Treatment

### Description of the Compositions Used

Dietary supplement (said second composition of the present invention): to be used in the first 20 days (first treatment stage) after the run-in period, has a composition of active ingredients (stock solution) based on (i) Artemisia vulgaris E.O. (400 mg/50 ml), (ii) Carum carvi E.O. (350 mg/50 ml), (iii) Lavender angustifolia, otherwise known as Lavandula officinalis (650 mg/50 ml) and (iv) Mentha piperita (600 mg/50 ml). Said dietary supplement was supplied in 10 ml single-dose containers containing a specific ready-to-use solution, prepared by mixing 15 drops of the stock solution with an appropriate fruit juice which assures suitable organoleptic characteristics. The association of the 4 active ingredients exerts an antibacterial, antifungal and antiparasitic/anthelmintic activity and can reduce, in a wholly natural manner, the intestinal concentration of several harmful microbial genera/species potentially associated with the onset and/or worsening of the symptoms of ALS, with particular reference to clostridia. The purpose of administering of said supplement was therefore to optimally prepare the intestinal environment before the treatment with the composition of the present invention.

The composition of the present invention used: to be used for 3 months (second treatment stage) following the treatment with the supplement. The composition is characterised by an action exerted by a specific muco-adherent gelling complex (EPS—natural exopolysaccharides produced in situ by the bacterial strain Streptococcus thermophilus ST10 deposited by Probiotical SpA with the Institute DSMZ in Germany on 19 Sep. 2011 and having the deposit number no. DSM 25246, once administered) capable of exerting a mechanical barrier effect extending to the whole gastrointestinal tract thanks to the complementary action of tara gum, if present, and of the exopolysaccharides (EPS) produced by the bacterial strain Streptococcus thermophilus ST10 deposited by Probiotical SpA with the Institute DSMZ in Germany on 19 Sep. 2011 and having the deposit number no. DSM 25246 which act, respectively, in the first and second halves of the intestinal tract. The gum is in fact gradually degraded by the resident microbiota during its progression in the lumen of the organ, whilst the secretion of EPS by S. thermophilus ST10 gradually takes on more relevance (2 billion viable cells per dose), thus eventually offering a total mechanical protection, capable of restoring the physiological barrier effect of the intestinal wall and preventing, in a wholly natural manner, the absorption of antigens, allergens and neurotoxins produced by the microbiota in the luminal compartment. The composition of the present invention further possesses a synergistic action of reinforcing the barrier effect described above, mediated by the presence of the microorganisms: Lactobacillus fermentum LF18 (optional), Lactobacillus fermentum LF19 (optional), Lactobacillus fermentum LF10 (4 billion viable cells per dose), Lactobacillus delbrueckii subsp. delbrueckii LDD01, Lactobacillus plantarum LP01 and Lactobacillus salivarius LS03 (2 billion viable cells per strain per dose). The total concentration of the strains (I) to (v) is 12 billion per dose.

### Dosage

Dietary supplement (first 20 days of the study): the patients were given 60 single-use containers pre-filled with 10 ml of an appropriate solution. The dosage is 3 containers a day per subject, to be taken at the end of the 3 main daily meals. Composition of the present invention (following 3 months): the patients received instructions to take two sachets a day for the first 30 days, and then to continue with one sachet a day for the following two months, to be taken in the morning and/or evening on an empty stomach, at least a half hour before a meal, after dissolving the contents of a sachet in half a glass of still water at room temperature.

At the end of the treatment protocol, the patients of the first group of subjects who received the composition of the present invention continued “sine die” the supplementation with a composition comprising the four or, optionally, six of the above-described microorganisms (I) to (VI) at a dosage of 1 billion viable cells of each strain per dose, without the presence of the components having a prevalently mechanical action, namely, the tara gum and exopolysaccharides—EPS—produced in situ by the strain S. thermophilus ST10, with the aim of maintaining the positive effects observed during the study. Whereas the second group of subjects (control group) began the protocol followed by the first group of subjects (treated group) if the results of the treatment were significant.

Four active ingredients of vegetable origin, (i) to (iv), were administered; they were specifically selected to reduce, in a wholly natural manner, the intestinal concentration of microbial groups and genera potentially implicated in the onset, persistence and/or worsening of the typical symptoms of ALS. In this manner it was also possible to enhance the effectiveness of the composition of the present invention administered in the following three months of treatment.

### Follow-Up

The patients were regularly followed up at monthly intervals for a 1 month run-in period, during the 20-day treatment with the supplement (t20), and during the treatment with the composition of the present invention. Subsequently at three-month intervals for at least 6 months (t50, t80, t110 and t200). On the occasion of each examination, the subjects underwent the assessment procedures, which comprised neurological evaluation including completion of the forms based on the ALS—FRS-R scale, spirometry and measurement of the forced vital capacity (FVC %) as well as completion of the ALSAQ-40 questionnaire with scales for measuring quality of life. All patients further underwent a dietary assessment which included: assessment based on medical history and objective assessment of the impairment in the ability to feed oneself autonomously, chewing and swallowing; assessment of weight loss compared to premorbid weight; dietary history (assessment of average calorie and protein intake over 24 hours); measurement of weight; and calculation of the body mass index (BMI).

Primary outcomes: Progression of disease measured with the ALS—FRS-R scale and FVC. Concentration of each intestinal bacterial species before and after treatment.

Secondary outcomes: Variations in nutritional state measured with the BMI. Variations in quality of life measured with the ALSAQ-40 scale.

### Results

As indicated in FIGS. 1 and 2, the administration of the composition according to the invention improves the course of the disease in human subjects both in terms of forced vital capacity (FVC) and in terms of disability assessed according to the ALS—FRS-R scale. FIG. 1 and FIG. 2 show an important technical effect provided by the treatment in accordance with the present invention on the course of the disease ALS. In Figure, 1 the improvement of the pathology is given by the increase (to a significant extent) of the parameter ALS—FRS-R in the first group of subjects (treated group) compared to the second group of subjects (control or placebo group). The same significant improvement was observed with reference to the parameter FVC in FIG. 2.

### Embodiments FRn of the Present Invention are the Following:

FR1. A composition comprising or, alternatively, consisting of an effective amount of a mixture which comprises at least one bacterial strain, for use in the preventive and/or curative treatment of symptoms and/or disorders connected to at least one neurodegenerative disease in a subject; wherein said use comprises the administration of said at least one bacterial strain belonging to the species Lactobacillus fermentum, Lactobacillus delbrueckii subsp. delbrueckii, Lactobacillus plantarum and Lactobacillus salivarius, and mixtures thereof.

FR2. The composition for use according to FR1, wherein said mixture comprises or, alternatively, consists of:
at least one bacterial strain belonging to the species Lactobacillus fermentum;
at least one bacterial strain belonging to the species Lactobacillus delbrueckii subsp. delbrueckii;
at least one bacterial strain belonging to the species Lactobacillus plantarum; and
at least one bacterial strain belonging to the species Lactobacillus salivarius.

FR3. The composition for use according to FR1 or FR2, wherein said mixture further comprises at least one bacterial strain belonging to the species Streptococcus thermophilus.

FR4. The composition for use according to any one of the preceding embodiments FR1-FR3, wherein the neurodegenerative disease is selected from among Alzheimer's disease, Parkinson's disease, Amyotrophic Lateral Sclerosis (ALS) or Lou Gehrig's disease, senile dementia, Huntington's disease, progressive supranuclear palsy, frontotemporal dementia and Lewy body dementia; it is preferably Amyotrophic Lateral Sclerosis (ALS).

FR5. The composition for use according to any one of the preceding embodiments FR1-FR4, wherein said mixture comprises or, alternatively, consists of an effective amount of the strain Lactobacillus fermentum LF10 deposited by Anidral Srl (now Probiotical SpA) with the Institute DSMZ in Germany on 20 Mar. 2007 and having the deposit number no. DSM 19187, Lactobacillus delbrueckii subsp. delbrueckii LDD01 deposited by Probiotical SpA with the Institute DSMZ in Germany on 10 Dec. 2008 and having the deposit number no. DSM 22106, Lactobacillus plantarum LP01 deposited by Mofin Srl with the Institute BCCM LMG in Belgium on 16 Oct. 2001 and having the deposit number no. LMG P-21021, Lactobacillus salivarius LS03 deposited by Probiotical SpA with the Institute DSMZ in Germany on 23 Jul. 2009 and having the deposit number no. DSM 22776 and, optionally, L. fermentum LF18 deposited by Probiotical SpA with the Institute DSMZ in Germany on 30 Jul. 2014 and having the deposit number no. DSM 29197 and L. fermentum LF19 deposited by Probiotical SpA with the Institute DSMZ in Germany on 30 Jul. 2014 and having the deposit number no. DSM 29198, and mixtures thereof.

FR6. The composition for use according to any one of the preceding embodiments FR1-FR5, wherein said mixture comprises the bacterial strain Streptococcus thermophilus ST10 deposited by Probiotical SpA with the Institute DSMZ in Germany on 19 Sep. 2011 and having the deposit number no. DSM 25246.

FR7. The composition for use according to any one of the preceding embodiments FR1-FR6, wherein the composition comprises a tara gum; the tara gum is preferably identified as E471.

FR8. The composition for use according to any one of the preceding embodiments FR1-FR7, wherein the administration takes place orally.

FR9. The composition for use according to any one of the preceding embodiments FR1-FR8, wherein the mixture comprises or, alternatively, consists of the bacterial strains Lactobacillus fermentum LF10 deposited by Anidral Srl (now Probiotical SpA) with the Institute DSMZ in Germany on 20 Mar. 2007 and having the deposit number no. DSM 19187, Lactobacillus delbrueckii subsp. delbrueckii LDD01 deposited by Probiotical SpA with the Institute DSMZ in Germany on 10 Dec. 2008 and having the deposit number no. DSM 22106, Lactobacillus plantarum LP01 deposited by Mofin Srl with the Institute BCCM LMG in Belgium on 16 Oct. 2001 and having the deposit number no. LMG P-21021 and Lactobacillus salivarius LS03 deposited by Probiotical SpA with the Institute DSMZ in Germany on 23 Jul. 2009 and having the deposit number no. DSM 22776 in a weight ratio of 1:1:1:1; and, optionally, L. fermentum LF18 deposited by Probiotical SpA with the Institute DSMZ in Germany on 30 Jul. 2014 and having the deposit number no. DSM 29197 and L. fermentum LF19 deposited by Probiotical SpA with the Institute DSMZ in Germany on 30 Jul. 2014 and having the deposit number no. DSM 29198, and mixtures thereof.

FR10. The composition for use according to any one of the preceding embodiments FR1-FR9, wherein said composition further comprises the bacterial strain Streptococcus thermophilus ST10 deposited by Probiotical SpA with the Institute DSMZ in Germany on 19 Sep. 2011 and having the deposit number no. DSM 25246 and a tara gum; the tara gum is preferably identified as E471.

1. A composition comprising or, alternatively, consisting of an effective amount of a mixture which comprises at least one bacterial strain, for use in the preventive and/or curative treatment of symptoms and/or disorders connected to at least one neurodegenerative disease in a subject; wherein said use comprises the administration of said at least one bacterial strain belonging to the species Lactobacillus fermentum, Lactobacillus delbrueckii subsp. delbrueckii, Lactobacillus plantarum and Lactobacillus salivarius, and mixtures thereof.

2. The composition for use according to claim 1, wherein said mixture comprises or, alternatively, consists of:
at least one bacterial strain belonging to the species Lactobacillus fermentum;
at least one bacterial strain belonging to the species Lactobacillus delbrueckii subsp. delbrueckii;
at least one bacterial strain belonging to the species Lactobacillus plantarum; and
at least one bacterial strain belonging to the species Lactobacillus salivarius.

3. The composition for use according to claim 1 or 2, wherein said mixture further comprises at least one bacterial strain belonging to the species Streptococcus thermophilus.

4. The composition for use according to any one of the preceding claims, wherein the neurodegenerative disease is selected from among Alzheimer's disease, Parkinson's disease, Amyotrophic Lateral Sclerosis (ALS) or Lou Gehrig's disease, senile dementia, Huntington's disease, progressive supranuclear palsy, frontotemporal dementia and Lewy body dementia; it is preferably Amyotrophic Lateral Sclerosis (ALS).

5. The composition for use according to any one of the preceding claims, wherein said mixture comprises or, alternatively, consists of an effective amount of the strain Lactobacillus fermentum LF10 deposited by Anidral Srl (now Probiotical SpA) with the Institute DSMZ in Germany on 20 Mar. 2007 and having the deposit number no. DSM 19187, Lactobacillus delbrueckii subsp. delbrueckii LDD01 deposited by Probiotical SpA with the Institute DSMZ in Germany on 10 Dec. 2008 and having the deposit number no. DSM 22106, Lactobacillus plantarum LP01 deposited by Mofin Srl with the Institute BCCM LMG in Belgium on 16 Oct. 2001 and having the deposit number no. LMG P-21021, Lactobacillus salivarius LS03 deposited by Probiotical SpA with the Institute DSMZ in Germany on 23 Jul. 2009 and having the deposit number no. DSM 22776 and, optionally, L. fermentum LF18 deposited by Probiotical SpA with the Institute DSMZ in Germany on 30 Jul. 2014 and having the deposit number no. DSM 29197 and L. fermentum LF19 deposited by Probiotical SpA with the Institute DSMZ in Germany on 30 Jul. 2014 and having the deposit number no. DSM 29198, and mixtures thereof.

6. The composition for use according to any one of the preceding claims, wherein said mixture comprises the bacterial strain Streptococcus thermophilus ST10 deposited by Probiotical SpA with the Institute DSMZ in Germany on 19 Sep. 2011 and having the deposit number no. DSM 25246.

7. The composition for use according to any one of the preceding claims, wherein the composition comprises a tara gum; the tara gum is preferably identified as E471.

8. The composition for use according to any one of the preceding claims, wherein the administration takes place orally.

9. The composition for use according to any one of the preceding claims, wherein the mixture comprises or, alternatively, consists of the bacterial strains Lactobacillus fermentum LF10 deposited by Anidral Srl (now Probiotical SpA) with the Institute DSMZ in Germany on 20 Mar. 2007 and having the deposit number no. DSM 19187, Lactobacillus delbrueckii subsp. delbrueckii LDD01 deposited by Probiotical SpA with the Institute DSMZ in Germany on 10 Dec. 2008 and having the deposit number no. DSM 22106, Lactobacillus plantarum LP01 deposited by Mofin Srl with the Institute BCCM LMG in Belgium on 16 Oct. 2001 and having the deposit number no. LMG P-21021 and Lactobacillus salivarius LS03 deposited by Probiotical SpA with the Institute DSMZ in Germany on 23 Jul. 2009 and having the deposit number no. DSM 22776 in a weight ratio of 1:1:1:1; and, optionally, L. fermentum LF18 deposited by Probiotical SpA with the Institute DSMZ in Germany on 30 Jul. 2014 and having the deposit number no. DSM 29197 and L. fermentum LF19 deposited by Probiotical SpA with the Institute DSMZ in Germany on 30 Jul. 2014 and having the deposit number no. DSM 29198, and mixtures thereof.

10. The composition for use according to any one of the preceding claims, wherein said composition further comprises the bacterial strain Streptococcus thermophilus ST10 deposited by Probiotical SpA with the Institute DSMZ in Germany on 19 Sep. 2011 and having the deposit number no. DSM 25246 and a tara gum; the tara gum is preferably identified as E471.
